(19) United States
(12) Patent Application Publication
(10) Pub. No.: US 2013/0095122 A1
(43) Pub. Date: Apr. 18, 2013

(54) Peptides or Antibodies Which Bind to Melanoma Inhibitory Activity (MIA) Protein

(71) Applicants: Anja Katrin Bosserhoff, Regensburg (DE); Burkhard König, Regensburg (DE); Alexander Riechers, Regensburg (DE); Jennifer Schmidt, Regensburg (DE)

(72) Inventors: Anja Katrin Bosserhoff, Regensburg (DE); Burkhard König, Regensburg (DE); Alexander Riechers, Regensburg (DE); Jennifer Schmidt, Regensburg (DE)

(21) Appl. No.: 13/583,541

(22) PCT Filed: Mar. 17, 2011
(86) PCT No.: PCT/EP2011/001338

§ 371 Date: Nov. 19, 2012

(30) Foreign Application Priority Data
Mar. 17, 2010 (EP) 10002807.5

(51) Int. Cl. C07K 5/097; C07K 5/087; C07K 7/06; C07K 5/107; C07K 16/18; C07K 7/08

(52) U.S. Cl. CPC C07K 5/0821; C07K 16/18; C07K 7/08; C07K 7/06; C07K 5/1016; C07K 5/0812
USPC 424/174.1; 530/389.7; 530/300; 530/331; 530/326; 530/329; 530/327; 530/330; 530/328; 530/391.1; 514/19.3; 514/17.1; 536/23.53; 536/23.1; 435/320.1; 435/344.1; 435/325; 514/44 R; 514/1.1; 530/409; 514/16.6; 514/16.8

(57) ABSTRACT

The present invention relates to peptides and antibodies which bind to melanoma inhibitory activity protein and the uses of such peptides and antibodies. The invention also relates to nucleic acids coding for such peptides or antibodies. The invention also relates to pharmaceutical compositions comprising such peptides or antibodies or such nucleic acids. The present invention also relates to small molecule compounds which bind to melanoma inhibitory activity protein and to uses of such small molecule compounds. Moreover, the present invention also relates to a method of preventing dimerization and/or aggregation of melanoma inhibitory activity (MIA) protein. The invention is based on the identification of the relevant sites of interaction of the MIA protein with the inhibitory peptides/antibodies. Considering the amino acid sequence of this protein deprived from the signalling peptide, the residues involved in the interaction are selected from: A7, K53, G54, R55, R57, L58, F59, V64, Y69, R85, D87, K91, and more preferably C17, S18, Y47, G61, G66, D67, L76, W102, D103, C106.

The present invention relates to peptides and antibodies which bind to melanoma inhibitory activity protein and to uses of such peptides and antibodies. The invention also relates to nucleic acids coding for such peptides or antibodies. The invention also relates to pharmaceutical compositions comprising such peptides or antibodies or such nucleic acids. The present invention also relates to small molecule compounds which bind to melanoma inhibitory activity protein and to uses of such small molecule compounds. Moreover, the present invention also relates to a method of preventing dimerization and/or aggregation of melanoma inhibitory activity (MIA) protein.

Malignant melanoma is characterized by aggressive local growth and early formation of metastasis. In order to identify autocrine growth-regulatory factors secreted by melanoma cells, melanoma inhibitory activity (MIA), an 11 kDa protein, strongly expressed and secreted by melanocytic tumor cells was purified from tissue culture supernatant of the human melanoma cell line HTZ-19.¹⁻² Today it serves as a reliable clinical serum tumor marker for detection of metastatic diseases and monitoring therapy responses of patients suffering from malignant melanoma. In addition, MIA plays an important functional role in melanoma development and cell invasion as its expression levels directly correlate with the capability of melanoma cells to form metastases in syngeneic animals.³⁻⁵.

After transcription, MIA mRNA is translated into a 131 amino acid precursor molecule and processed into a mature protein consisting of 107 amino acids after cleavage of the secretion signal sequence.² The transport of MIA protein to the cell rear is induced after migratory stimuli.⁶ Following secretion, MIA subsequently binds to cell adhesion receptors integrin α₄β₁ and integrin α₅β₁. In addition, MIA masks their binding sites at ECM (extracellular matrix) molecules including fibronectin, laminin and tenascin.^{3,7} Consequently, cell adhesion contacts are reduced, enabling tumor cells to migrate and invade into healthy tissue, resulting in enhanced metastatic potential.

Previously, the three-dimensional structure of MIA protein was solved by multidimensional nuclear magnetic resonance (NMR) spectroscopy and X-ray crystallography techniques.⁸⁻¹² Corresponding data indicated that MIA defines a novel type of secreted protein comprising an SH3 domain like fold.

US 2006/0128607 describes a number of peptides that appear to inhibit the activity of MIA protein. The peptides reported therein are believed to bind to MIA protein and to thereby prevent its binding to other non-MIA proteins. Two examples of these peptides are indicated in the present application as SEQ ID NO:46 and 47, corresponding to SEQ ID NO: 46 and 48 of US 2006/0128607. The peptides of US 2006/0128607 are reported to bind to MIA through individual residues via hydrogen-bonding and van der Waals contacts. Although details with respect to individual amino acid residues are given, US 2006/0128607 does not describe the overall effect that binding of the peptides has on MIA protein.

Accordingly, it was an object of the present invention to provide for alternative molecules, such as peptides or antibodies or small molecule mimetics that also bind to MIA protein. It was also an object of the present invention to provide for peptides or antibodies or small molecule mimetics that interact with MIA protein and prevent its dimerization and/or aggregation.

The objects of the present invention are solved by a peptide or antibody which binds to melanoma inhibitory activity (MIA) protein and prevents dimerization and/or aggregation thereof, which peptide is not SEQ ID NO:46 or 47.

In one embodiment, binding thereof to MIA protein occurs at a surface of said MIA protein formed by at least three amino acid residues of said MIA protein selected from cysteine 17, serine 18, tyrosine 47, glycine 61, glycine 66, aspartate 67, leucine 76, tryptophan 102, aspartate 103, cysteine 106, valine 64, tyrosine 69, aspartate 87, lysine 91, glycine 54, leucine 58, phenylalanine 59, alanine 7, lysine 53, arginine 55, arginine 57, arginine 85 and lysine 94. Preferred residues are selected from cysteine 17, serine 18, tyrosine 47, glycine 61, glycine 66, aspartate 67, leucine 76, tryptophan 102, aspartate 103, cysteine 106, alanine 7, lysine 53, arginine 55, arginine 57, arginine 85, and lysine 94. Particularly preferred residues are cysteine 17, serine 18, tyrosine 47, glycine 61, glycine 66, aspartate 67, leucine 76, tryptophan 102, aspartate 103 and cysteine 106.

It should be noted that the numbering of the amino acid residues indicated above refers to the position of the respective amino acid residue within the sequence of MIA protein. The amino acid sequence of MIA protein, as used herein, is indicated in SEQ ID NO:48 (see further below).

In one embodiment, binding thereof to MIA protein is measured by a heterogeneous transition metal-based fluorescence polarization (HTFP) assay, wherein, preferably, binding of said peptide to MIA protein is indicated by a ratio P/P₀, wherein P is the fluorescence polarization signal of an MIA protein labeled with a luminescent transition metal complex in the presence of a substrate-bound MIA protein and in the presence of said peptide or antibody, and P₀ is the fluorescence polarization signal of free MIA protein labeled with said luminescent transition metal complex in the absence of a substrate bound MIA protein and in the absence of said peptide or antibody, wherein the ratio P/P₀ of said peptide or antibody, when determined in a heterogeneous transition metal-based fluorescence polarization (HTFP) assay at a defined concentration of said peptide or antibody, is smaller than P/P₀ of the peptide having the amino acid sequence of SEQ ID NO:47, said P/P₀ of said SEQ ID NO:47 peptide having been determined in a HTFP assay at the same defined peptide concentration. In another embodiment, binding of said peptide to MIA protein is determined by NMR, preferably heteronuclear NMR, more preferably ¹⁵N-¹H-HSQC-NMR.

In one embodiment, the peptide according to the present invention has an amino acid sequence selected from SEQ ID NO:1-45, preferably an amino acid sequence selected from SEQ ID NO:1-9. In one embodiment, the antibody according to the present invention includes a region having an amino acid sequence selected from SEQ ID NO:1-45, preferably an amino acid sequence selected from SEQ ID NO:1-9.

The objects of the present invention are also solved by a peptide having an amino acid sequence selected from SEQ ID NO:1-45, in particular SEQ ID NO:1-9, or by an antibody including a region having an amino acid sequence selected from SEQ ID NO:1-45, preferably an amino acid sequence selected from SEQ ID NO:1-9. The term “a peptide having an amino acid sequence selected from SEQ ID NO:1-45” or “ . . . SEQ ID NO: 1-9” is meant to refer to a peptide which consists of the amino acid sequence selected from SEQ ID NO:1-45 or from SEQ ID NO: 1-9.

In one embodiment, the peptide or antibody according to the present invention is amidated at its C-terminus or is pegylated.

The objects of the present invention are solved by the peptide or antibody according to the present invention for use in the treatment of cancer.

In one embodiment, said cancer is selected from melanoma, chondrosarcoma, mamma carcinoma and colon carcinoma.

The objects of the present invention are solved by the peptide or antibody according to the present invention for use in the prevention of metastasis of said cancer.

The objects of the present invention are solved by the peptide or antibody according to the present invention for use in the treatment of a degenerative disorder of cartilage.

In one embodiment, said degenerative disorder of cartilage is selected from rheumatoid arthritis, degeneration of cartilage in a joint, degenerative disc disease, meniscus tears, anterior crucial ligament (ACL) injury, arthritis, osteoarthritis, psoriatic arthritis, juvenile chronic arthritis, rhizomelic arthritis, rheumatoid poly-arthritis, synovitis and villonodular synovitis.

The objects of the present invention are solved by the peptide or antibody according to the present invention for use in binding to MIA protein and/or preventing dimerization and/or aggregation of MIA protein.

The objects of the present invention are solved by a nucleic acid coding for the peptide or antibody according to the present invention.

The objects of the present invention are solved by a vector or construct comprising the nucleic acid according to the present invention.

The objects of the present invention are solved by a cell or tissue comprising the nucleic acid according to the present invention or the vector or construct according to the present invention.

The objects of the present invention are solved by a pharmaceutical composition comprising the peptide or antibody according to the present invention or the nucleic acid according to the present invention or the vector or construct according to the present invention or the cell or tissue according to the present invention, and a suitable pharmaceutically acceptable carrier.

The objects of the present invention are also solved by a method of treatment of a cancer, said method comprising administration of the peptide or antibody according to the present invention to a patient having a cancer. In one embodiment, said cancer is selected from melanoma, chondrosarcoma, mamma carcinoma and colon carcinoma. In one embodiment, said method of treatment is particularly aimed at the prevention of metastasis of said cancer, in particular one of the foregoing cancers.

The objects of the present invention are also solved by a method of treatment of a degenerative disorder of cartilage, said method comprising administration of the peptide or antibody according to the present invention to a patient having a degenerative disorder of cartilage. In one embodiment, said degenerative disorder of cartilage is selected from rheumatoid arthritis, degeneration of cartilage in a joint, degenerative disc disease, meniscus tears, anterior crucial ligament (ACL) injury, arthritis, osteoarthritis, psoriatic arthritis, juvenile chronic arthritis, rhizomelic arthritis, rheumatoid polyarthritis, synovitis and villonodular synovitis.

The objects of the present invention are also solved by the use of a peptide or antibody according to the present invention for binding to MIA protein and/or preventing dimerization and/or aggregation thereof. In one embodiment, such use is an in-vitro-use. In another embodiment, such use is an in-vivo-use.

The objects of the present invention are also solved by a method of preventing dimerization and/or aggregation of melanoma inhibitory activity (MIA) protein, said method comprising:
exposing a MIA protein to a compound which selectively interacts with and/or binds to a surface of said MIA protein formed by at least three amino acid residues of said MIA protein, said at least three amino acid residues being selected from cysteine 17, serine 18, tyrosine 47, glycine 61, glycine 66, aspartate 67, leucine 76, tryptophan 102, aspartate 103, cysteine 106, valine 64, tyrosine 69, aspartate 87, lysine 91, glycine 54, leucine 58, phenylalanine 59, alanine 7, lysine 53, arginine 55, arginine 57, arginine 85, and lysine 94, preferably cysteine 17, serine 18, tyrosine 47, glycine 61, glycine 66, aspartate 67, leucine 76, tryptophan 102, aspartate 103, cysteine 106, alanine 7, lysine 53, arginine 55, arginine 57, arginine 85 and lysine 94, more preferably cysteine 17, serine 18, tyrosine 47, glycine 61, glycine 66, aspartate 67, leucine 76, tryptophan 102, aspartate 103 and cysteine 106.

It should be noted that the numbering of the amino acid residues indicated above refers to the position of the respective amino acid residue within the sequence of MIA protein. The amino acid sequence of MIA protein, as used herein, is indicated in SEQ ID NO:48 (see further below).

In one embodiment of this method, said compound is a peptide, an antibody or a small molecule compound.

In one embodiment said peptide has an amino acid sequence which is not SEQ ID NO:46 or 47.

In one embodiment, said peptide has an amino acid sequence selected from SEQ ID NO: 1-45, preferably SEQ ID NO: 1-9.

In one embodiment, said peptide is amidated at its C-terminus or is pegylated.

In one embodiment, said antibody is a monoclonal antibody or a polyclonal antibody.

In one embodiment, said small molecule compound is obtained from a combinatorial chemistry library.

In one embodiment, said method is an in-vitro-method.

The term “preventing dimerization of MIA protein” is meant to refer to both a situation where the formation of a dimer of MIA protein is prevented, and a situation wherein a dimer, after its formation, is subsequently dissociated again. Both situations are meant to be encompassed by the term “prevention of dimerization of MIA protein”. The term “dimerization” is also meant to encompass the formation of multimers of MIA protein, involving more than two MIA monomers. It is also meant to encompass the formation of aggregates of MIA protein. “Multimers” involve three or four or five etc. or more or a plurality of MIA monomers.

The present inventors have surprisingly found that MIA forms a dimer or multimer and that a number of peptides and antibodies strongly interact with MIA protein and thereby prevent its dimerization and/or aggregation. This becomes particularly evident in a heterogeneous transition metal-based fluorescence polarization assay (HTFP assay), wherein the ratio P/P₀ is measured. P is the fluorescence polarization signal of a MIA protein labeled with a transition metal complex in the presence of substrate bound MIA-protein and of the peptide or antibody to be tested. P₀ is the fluorescence polarization signal of free MIA-protein labeled with said luminescent transition metal complex in the absence of substrate bound MIA-protein and in the absence of said peptide or antibody. In the absence of the peptide or antibody, usually, the labeled MIA-protein would interact with the substrate bound MIA-protein, which, in turn, would contribute to a reduction in rotational mobility of the labeled MIA-protein, and therefore, the fluorescence polarization signal would increase upon such interaction. If, additionally, a peptide or antibody is present that interferes with such interaction, no or little dimerization/aggregation occurs and no or little increase in fluorescence polarization signal would be detected. The smaller or even more negative P/P₀ is, the stronger such interference with dimer formation and aggregation is, and the better such peptide or antibody prevents dimerization/aggregation of MIA protein. The inventors have identified the residues in the MIA sequence (SEQ ID NO:48) which are involved in the binding of said peptides to MIA.

As outlined further below, the MIA dimer is characterized by a head-to-tail-orientation with the dimerization domains comprising the n-Src loop and the cleft next to the distal loop. The interface between two monomers is, in one monomer, formed by at least three amino acid residues of the amino acid sequence of MIA protein, said at least three amino acid residues being selected from cysteine 17, serine 18, tyrosine 47, glycine 61, glycine 66, aspartate 67, leucine 67, tryptophan 102, aspartate 103, cysteine 106, valine 64, tyrosine 69, aspartate 87 and lysine 91. The preferred amino acid residues, in this monomer, are selected from cysteine 17, serine 18, tyrosine 47, glycine 61, glycine 66, aspartate 67, leucine 76, tryptophan 102, aspartate 103 and cysteine 106. In the other monomer that participates in the dimerization, the interface is formed by at least three amino acid residues of the sequence of MIA protein selected from alanine 7, lysine 53, arginine 55, arginine 57, arginine 85, lysine 94, glycine 54, leucine 58 and phenylalanine 59. Preferred residues in this context are at least three residues selected from alanine 7, lysine 53, arginine 55, arginine 57, arginine 85 and lysine 94.

In preferred embodiments, P/P₀ of the peptides or antibodies according to the present invention, for a given peptide concentration, is smaller or more negative than P/P₀, determined for SEQ ID NO:46 or SEQ ID NO:47.

Particularly preferred peptides are SEQ ID NO:1-45, more preferably SEQ ID NO:1-9.

The peptides and antibodies in accordance with the present invention or the nucleic acids coding therefore may form part of a pharmaceutical composition. The formulation of such pharmaceutical compositions is known to someone skilled in the art and can be formulated using an appropriate pharmaceutically acceptable carrier. The peptides and antibodies in accordance with the present invention may also be combined and/or formulated and/or administered together with agents selected from a) immunostimulatory agents, such as interleukin-2, interferon-alpha, interferon-gamma, interleukin-12, GM-CSF, b) chemotherapeutic agents, such as Taxanes, Taxotere, Temoda, Anthracyclines, Vinca Alkaloids, c) gene-therapeutic agents suitable for gene-transfer, such as interleukin-7, 2, 4, 12, interferon-gamma, HSV-TK (Herpes-Simplex-virus thymidine-kinase), d) antiangiogenic and/or anti-invasive agents, and e) vaccines.

The peptides and antibodies in accordance with the present invention may also be pegylated. Such pegylation is known to a person skilled in the art and can be performed in accordance with standard laboratory procedures, as for example described by Morar et al., BioPharm International, 2006, 19 (4), and Harris, et al., Clin. Pharmacokinet. 2001, 40:539-551.

The peptides and antibodies in accordance with the present invention may also be amidated, preferably at their C-terminus. Such amidation may be the result of the synthesis of the peptides, using solid-phase-synthesis, or the peptides may be amidated using enzymatic reactions or simple chemical synthesis methods, such as are for example described in Chang et al., Bioconjugate Chem., 2009, 20 (2), pp. 197-200.

Also encompassed by the present invention are nucleic acids coding for the peptides and anti-bodies according to the present invention. In one embodiment, the peptides and anti-bodies according to the present invention are administered as protein compounds, i.e. peptides and antibodies. In another embodiment, the peptides and antibodies according to the present invention are administered as their corresponding nucleic acids for subsequent expression of the peptides and antibodies according to the present invention. When the peptides and antibodies in accordance with the present invention are administered, they are typically administered at a concentration range of 0.1 μg/kg body weight to 1 g/kg body weight, preferably from 1 μg/kg body weight to 1 mg/kg body weight, more preferably from 1 μg/kg body weight to 100 μg/kg body weight.

The antibodies in accordance with the present invention may be monoclonal or polyclonal antibodies. They are produced by methods known to someone skilled in the art, such as for example by injecting the antigen, in this case MIA or epitopes thereof into a mammal to obtain quantities of polyclonal antibodies from the blood isolated from these animals. Likewise, to obtain monoclonal antibodies, antibody-secreting lymphocytes are isolated from such animal and immortalized by fusing them with a cancer cell line to produce a hybridoma which will continually grow and secrete antibodies in culture. Single hybridoma cells may be isolated by dilution cloning to generate cell clones that all produce the same monoclonal antibody. Appropriate antibodies that specifically bind to the interface of MIA dimers may be selected by generating antibodies using both wildtype MIA protein as well as MIA-mutants. If a MIA mutant, i.e. mutation at a particular residue of MIA, affects the formation of the dimer, for example because the mutation lies in the dimer interface, the resultant antibody generated therewith, is likely not to interact with dimer formation in the wildtype MIA, and the residue identified by such mutation is a residue involved in dimer formation.

Antibodies which are selective for interfering with dimer formation can be selected by first incubating the prepared antibodies with wt MIA immobilized on a suitable carrier, for example sepharose, and subsequent removal of all antibodies that do not bind to the immobilized wt MIA. In a second step, antibodies bound to the immobilized wt MIA will be eluted and subsequently incubated with a similarly immobilized MIA mutant which is unable to dimerize. Antibodies selective for the dimerization domain will then remain unbound in the supernatant, while antibodies targeting other domains will bind to the immobilized mutant of MIA.

Also encompassed by the present invention are small molecule mimetic compounds which are non-peptidic in nature and which may be derived from combinatorial chemistry libraries which are commercially available. Also such small molecule mimetic compounds may be used to prevent dimerization and/or aggregation of MIA protein. Preferably, a “small molecule mimetic compound” or “small molecule compound”, as used herein, refers to a non-proteinaceous compound having a molecular weight <2000, more preferably <1000.

In the following, reference is made to the figures, wherein the figures show the following:

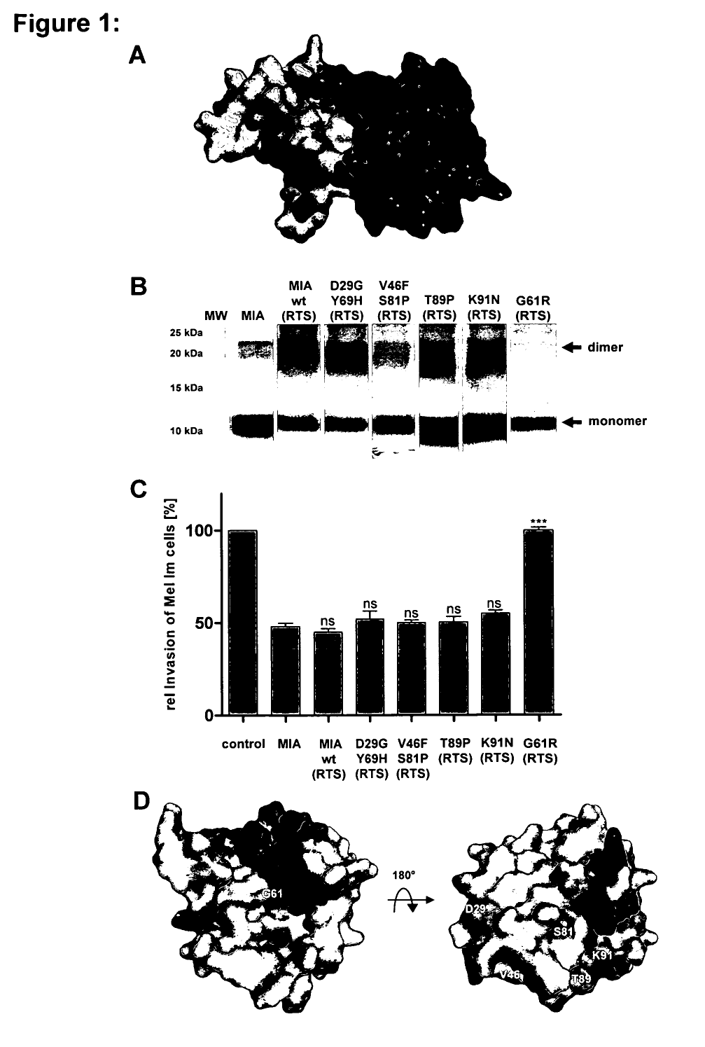

FIG. 1 shows MIA protein is functionally inactive as a monomer. (A) Structure of the MIA dimer according to shape complementarity analyses. The MIA dimer is characterized by a head-to-tail orientation, with the dimerization domains consisting of the n-Src loop and the cleft next to the distal loop. (B) Western blot analysis of MIA assessing their ability to form dimers. The first lane shows recombinant wt MIA, followed by the same protein in an unpurified RTS expression system (wt) and mutants D29G/Y69H, V46F/S81P, T89P, K91N and G61R. All homologues, except for G61R, clearly show a dimer band. (C) Correlation between dimerization and functional activity revealed that all MIA mutants capable to dimerize are functionally active in Boyden chamber invasion assays as reflected by a reduction in the number of invaded cells due to interference with cell adhesion. Mutant G61R, which does not form protein dimers does not show any MIA induced effect. (D) NMR structure of MIA showing the dimerization domains and the mutation sites. The dimerization domains in the n-Src loop and next to the distal loop are depicted in darker grey tones, respectively. Mutation sites which do not influence dimerization and functional activity are shown by the individual labels of individual amino acids (D29, V46, S81, T89, K91) and obviously lie outside the dimerization domains. The site of mutation G61R, which is in direct contact with the dimerization domain next to the distal loop, is shown by G61. This figure was generated using PyMol (Delano, W. L., The PyMol Molecular Graphics System (2002) Delano Scientific, Palo Alto, Calif., USA).

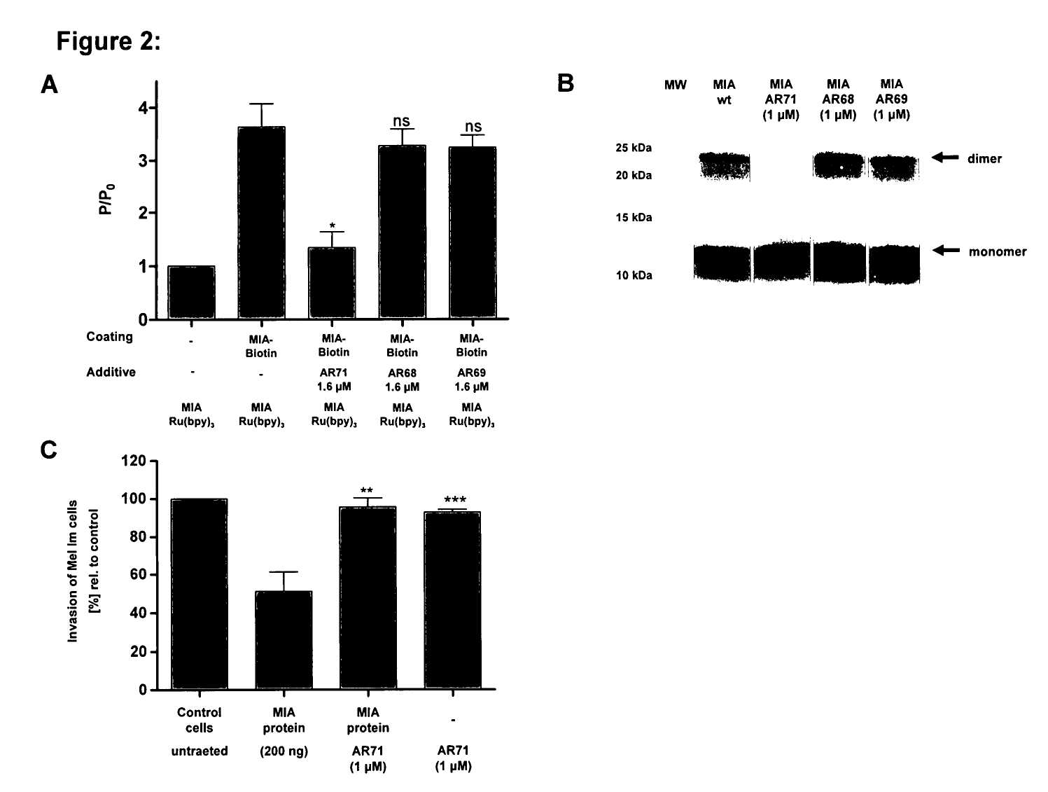

FIG. 2 shows that Peptide SEQ ID NO:47 (=AR71) prevents MIA dimerization. (A) Heterogeneous transition-metal based fluorescence polarization (HTFP) assay for probing AR71 for its ability to directly interfere with MIA-MIA interaction. In the control lanes the FP signal of MIA-Ru(bpy)₃ was measured in a well coated with MIA-biotin compared to an uncoated well. The significant increase in FP in the well coated with MIA-biotin indicates binding of MIA-Ru(bpy)₃ to the immobilized MIA-biotin. The binding of MIA-inhibitory compound AR71 promotes dissociation of MIA dimers and displaces the surface-bound MIA-Ru(bpy)₃, as reflected by a decrease in fluorescence polarization signal. Peptides AR68 and AR69, which serve as negative controls also derived from phage display, do not interfere with MIA-MIA interaction. (B) Western Blot analysis of MIA incubated with 1 μM AR71 demonstrates peptide-induced dissociation of the dimer, as deduced by a strong reduction of the dimer bands compared to the control lane. MIA-binding peptides AR68 and AR69 do not lead to reduced dimer formation. (C) Boyden chamber invasion assays using the human melanoma cell line Mel Im indicate that AR71 almost completely inhibits MIA activity. Interference of MIA with cell attachment to matrigel results in a decrease in cell invasion; after external treatment with MIA invasion of Mel Im cells is significantly reduced about 40% to 50% compared to untreated control cells. Pre-incubation of MIA with the respective inhibitory peptide results in a complete neutralization of the MIA effect. The two control lanes confirm that AR71 alone does not influence the migratory behaviour since exposure of cells to the peptide in absence of MIA does not alter the quantity of migrated cells.

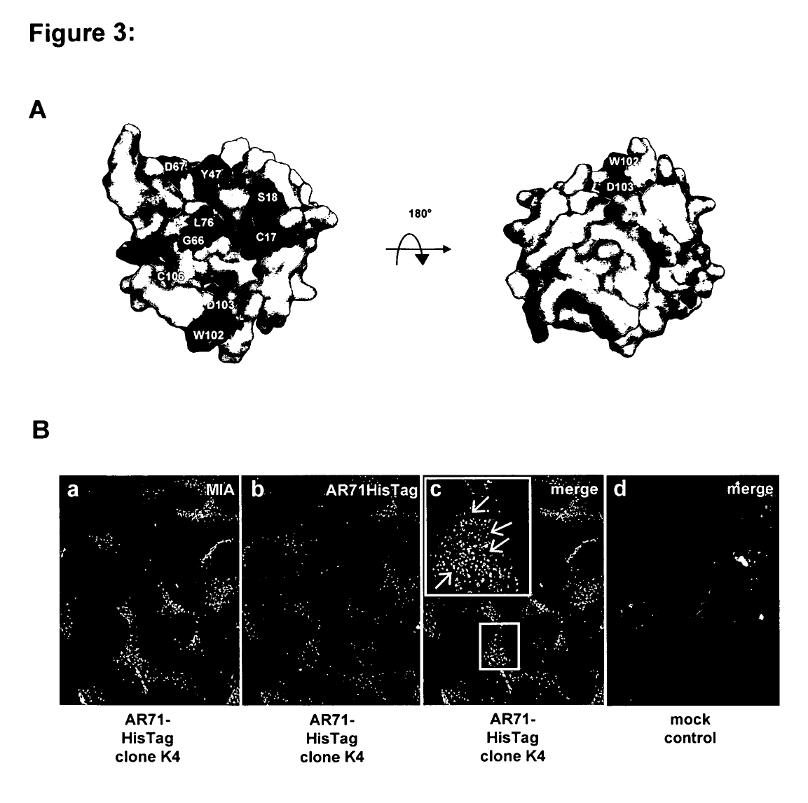

FIG. 3 shows Chemical shift differences of MIA upon titration with the dodecapeptide AR71 (=SEQ ID NO:47). (A) Most significant chemical shift differences projected onto the van der Waals surface of MIA upon titration with the peptide AR71 are shown by the respective amino acid residue labelled with their respective one-letter code and residue number. The binding site is located in the dimerization domain next to the distal loop (compare FIG. 1D). This figure was generated using PyMol (Delano, W. L., The PyMol Molecular Graphics System (2002) Delano Scientific, Palo Alto, Calif., USA). (B) Immunofluorescence studies of murine B16 melanoma cells stably transfected with a (Sig)-AR71-HisTag construct. While a) shows MIA (FITC) and b) displays AR71-HisTag, colocalization is indicated by white arrows in c). d) Corresponding mock control without AR71-HisTag.

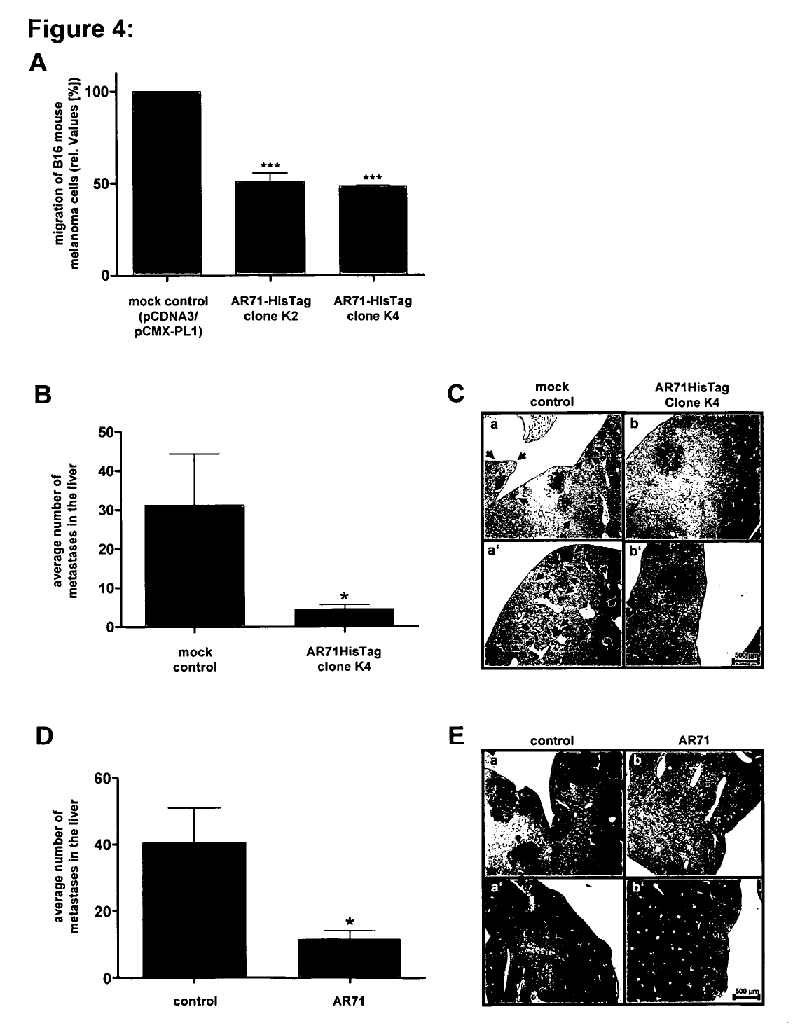

FIG. 4 shows the Effect of MIA inhibitory peptide AR71 (=SEQ ID NO:47) on formation of metastases in vivo. (A) Murine B16 melanoma cells stably transfected with a (secretion-signal)-AR71-HisTag containing construct were analyzed for their migratory activity in a Boyden chamber assay. Compared to the mock control, migration is drastically reduced in the two Sig-AR71-HisTag expressing cell clones clone K2 and clone K4. (B) Sig-AR71-HisTag clone K4 as well as a corresponding mock control were injected into the spleen of Bl/6N mice, respectively. Histological analysis of haematoxylin and eosin stained liver sections revealed that mice being injected with Sig-AR71-HisTag clones comprised significantly fewer metastases than the mock control. (C) Representative histological liver sections (hematoxylin and eosin stained), two of mice injected with the B16 mock control (a and a′) and two of mice injected with the Sig-AR71-HisTag expressing cell clone K4 (b and b′). Black arrows indicate small metastases. (D) Wild type murine B16 melanoma cells were injected into the spleen of Bl/6N mice with the mice being subsequently treated with i.v. injections of AR71 (50 μg every 24 h). Histological analyses revealed a significant reduction of the average number of metastases in the liver of mice treated with AR71 compared to the liver of untreated control mice. (E) Representative histological liver sections (hematoxylin and eosin stained), two of untreated (a and a′) and two of treated mice (b and b′).

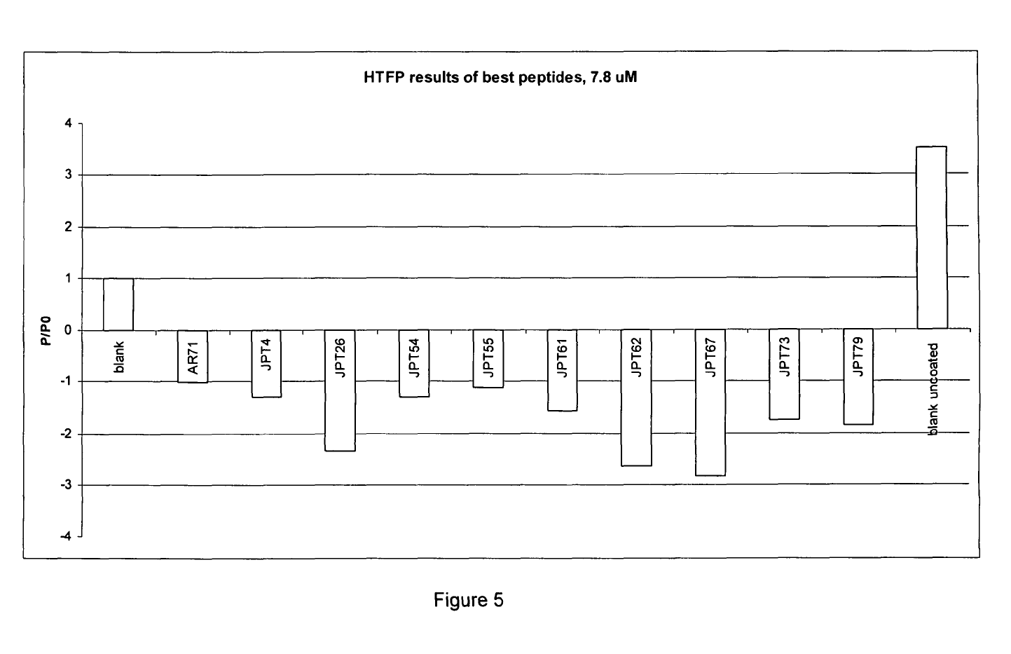

FIG. 5 shows that peptides SEQ ID NO:1-9 prevent MIA-protein dimerization: The graph shows a heterogeneous transition-metal based fluorescence polarization (HTFP) assay for probing SEQ ID NO:1-9, for their ability to directly interfere with MIA-MIA interaction. In the control lanes, the fluorescence polarization signal (FP signal) of MIA-Ru(bpy)₃ (bpy=bispyridyl) was measured in a well coated with MIA-biotin compared to an uncoated well. The significant increase in FP in the well coated with MIA-biotin indicates binding of MIA-Ru (bpy)₃ to the immobilized MIA-biotin. The binding of a MIA-inhibitory peptide promotes dissociation of MIA protein dimers and displaces the surface-bound MIA-Ru(bpy)₃ reflected by a decrease in fluorescence polarization signal. The term “blank” refers to a well with MIA-biotin and without a peptide, whereas the term “blank uncoated” refers to a well without MIA-biotin and without a peptide.

The individual peptide sequences shown in FIG. 5 are as follows, with reference to the enclosed sequence listing: (It should be noted that the sequences, although shown here un-pegylated and not amidated, may also be pegylated and/or amidated; and thus also encompassed by the present invention.)

JPT67 is SEQ ID NO:1, JPT62 is SEQ ID NO:2, JPT71 is SEQ ID NO:41, JPT26 is SEQ ID NO:3, JPT79 is SEQ ID NO:4, JPT73 is SEQ ID NO:5, JPT61 is SEQ ID NO:6, JPT54 is SEQ ID NO:7, JPT4 is SEQ ID NO:8, JPT55 is SEQ ID NO:9, AR71 is SEQ ID NO:47.

Moreover, reference is made to the sequences in the enclosed sequence listing, wherein SEQ ID NO:1-45 are peptide sequences in accordance with the present invention, and

SEQ ID NO: 46
is peptide FHWHPRLWPLPS,
SEQ ID NO: 47
is peptide FHWRYPLPLPGQ.

Sometimes, these two peptides are also herein referred to as “AR70” and “AR71”, respectively.

SEQ ID NO:48 is the amino acid sequence of the mature MIA protein (monomer).

More specifically, the following overview lists the respective sequences:

| SEQ ID NO: | Sequence: | Other designation: |
|---|---|---|
| 1 | WHF | JPT67 |
| 2 | FHWRYPLPLPGQHHHHHH | JPT62 |
| 3 | WHWRYP | JPT26 |
| 4 | FHWRYPDPDPGQ | JPT79 |
| 5 | WWW | JPT73 |
| 6 | HHHHHHFHWRYPLPLPGQ | JPT61 |
| 7 | WHW | JPT54 |
| 8 | FHWRYPGPGPGQ | JPT4 |
| 9 | FHWH | JPT55 |
| 10 | KGRGRLFW | |
| 11 | FHW | |
| 12 | FHRRYPDPDPGQ | |
| 13 | FHWWYP | |
| 14 | FHWRYPGPLPGQ | |
| 15 | FHWRYPLPGPGQ | |
| 16 | FHWRYPDPLPGQ | |
| 17 | FHWRYPLPDPGQ | |
| 18 | AHWRYPLPLPGQ | |
| 19 | FAWRYPLPLPGQ | |
| 20 | FWWRYPLPLPGQ | |
| 21 | FHWGYPLPLPGQ | |
| 22 | FHWEYPLPLPGQ | |
| 23 | FHWRYALPLPGQ | |
| 24 | FHWRYPLALPGQ | |
| 25 | FHWRYPLPLAGQ | |
| 26 | FHWRYP | |
| 27 | FHWRAPLPLPGQ | |
| 28 | FHWRYPLPLPGR | |
| 29 | FRWRYPLPLPGQ | |
| 30 | RFHWRYP | |
| 31 | FHWRYPLPLPG | |
| 32 | FHWRYPLP | |
| 33 | FHPRYPL | |
| 34 | FFW | |
| 35 | WFF | |
| 36 | WFW | |
| 37 | WFH | |
| 38 | FHF | |
| 39 | HHF | |
| 40 | FHH | |
| 41 | HHH | JPT71 |
| 42 | WWF | |
| 43 | WWH | |
| 44 | HWW | |
| 45 | FWW | |
| 46 | FHWHPRLWPLPS | AR70 |
| 47 | FHWRYPLPLPGQ | AR71 |
| 48 | GPMPKLADRKLCADQECSHPISMAVALQDYMAPDCRFLTIHRG | |
| | QVVYVFSKLKGRGRLFWGGSVQGDYYGDLAARLGYFPSSIVRE | |
| | DQTLKPGKVD VKTDKWDFYCQ | |

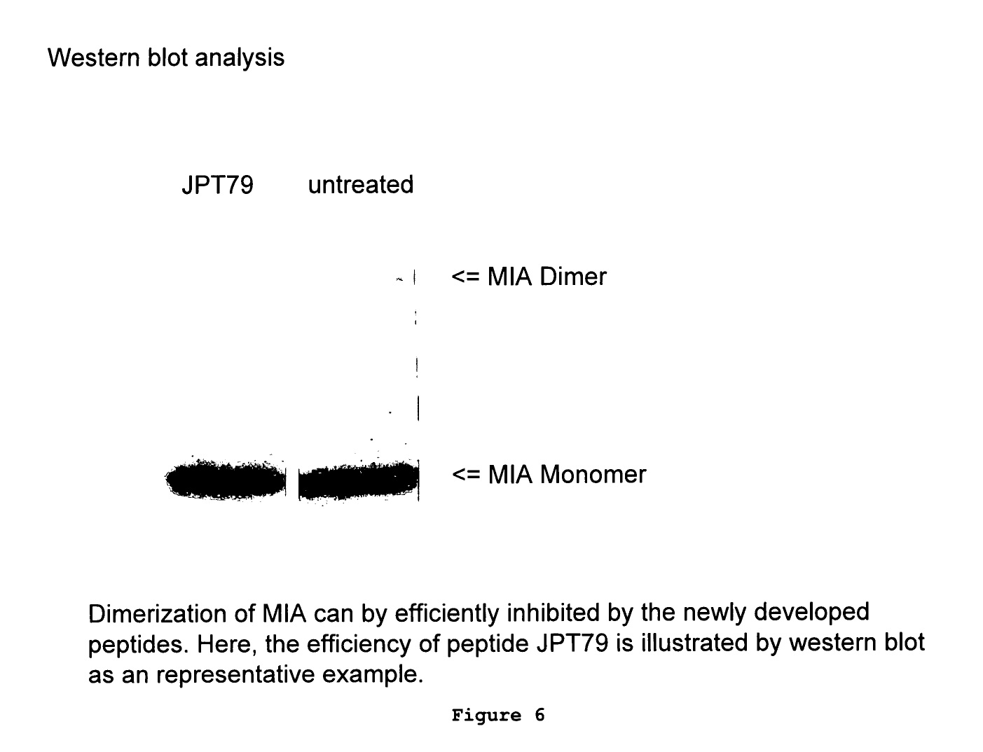

FIG. 6 shows that dimerization of MIA can be efficiently inhibited by the peptides in accordance with the present invention. In FIG. 6, the efficiency of peptide JPT79 (SEQ ID NO:4) is illustrated by western blotting as a representative example.

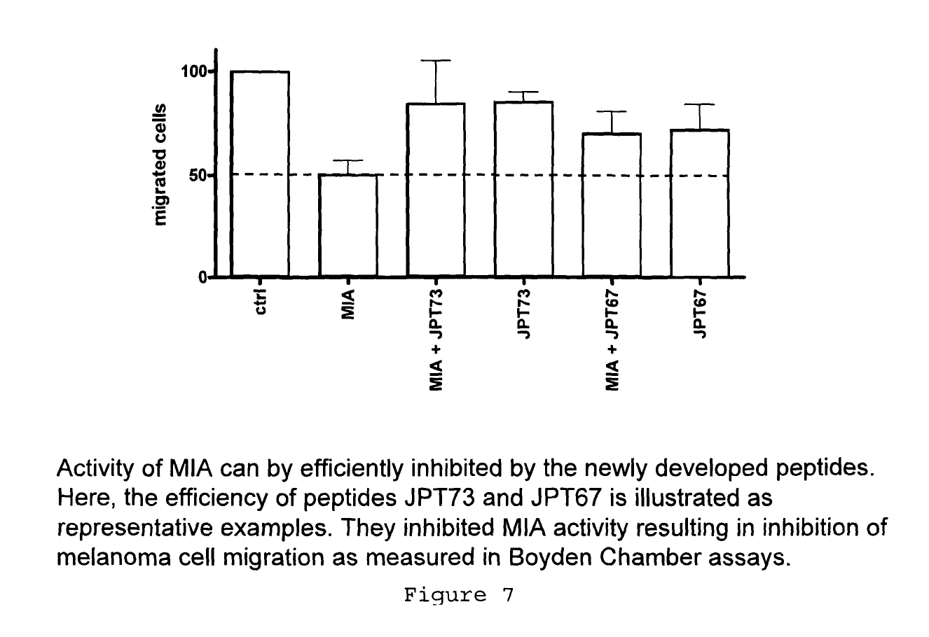

FIG. 7 shows that the activity of MIA can be efficiently inhibited by the peptides in accordance with the present invention. As an example, peptides JPT73 (SEQ ID NO:5) and JPT67 (SEQ ID NO:1) inhibit MIA-activity, as measured in Boyden Chamber assays. Interference of MIA with cell attachment to matrigel results in a decrease in cell invasion; after external treatment with MIA invasion of Mel Im cells is significantly reduced about 40% to 50% compared to untreated control cells. Pre-incubation of MIA with the respective inhibitory peptide results in a neutralization of the MIA effect.

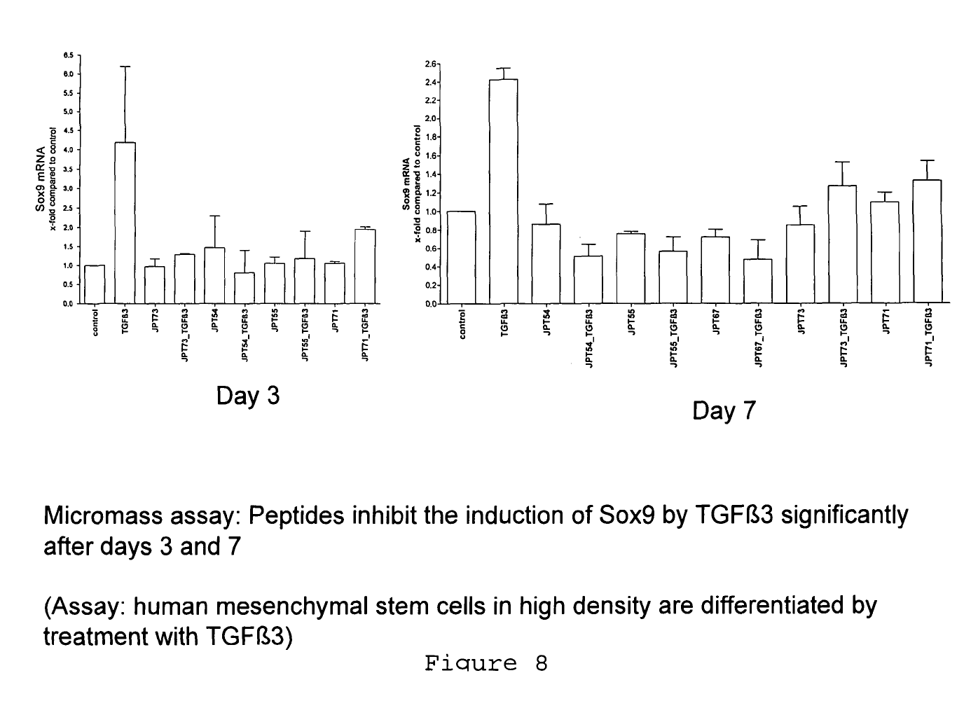

FIG. 8 shows that the peptides in accordance with the present invention inhibit the induction of Sox9 mRNA by TGFβ3 significantly after days 3 and 7. Peptide JPT71 in this figure corresponds to SEQ ID NO: 41. More specifically, in this micromass assay, Sox9 expression as marker for chondrocytic differentiation is induced after treatment of the cells with TGFβ3. MIA is an important regulator of chondrogenic differentiation after induction by TGFβ3. Using the inhibitory peptides, chondrocytic differentiation by TGFβ3 was strongly inhibited confirming the strong effect of the peptides on MIA activity.

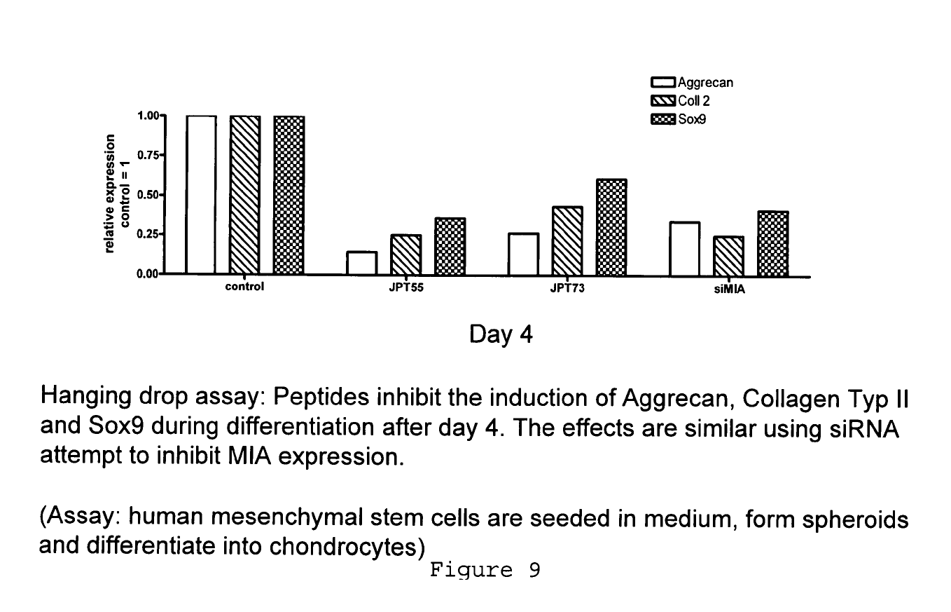

FIG. 9 shows the results of a hanging drop assay. Accordingly, the peptides in accordance with the present invention, inhibit the induction of Aggrecan, collagen type II and Sox9 during differentiation after day 4. The effects observed for the peptides in accordance with the present invention, in this example JPT55 (SEQ ID NO:9) and JPT73 (SEQ ID NO:5), are similar to those results obtained when using siRNA to inhibit MIA expression. In the assay, in agreement with the data shown in FIG. 8, the strong potential of the peptides to inhibit chondrogenic differentiation by inhibiting MIA activity was underlined.

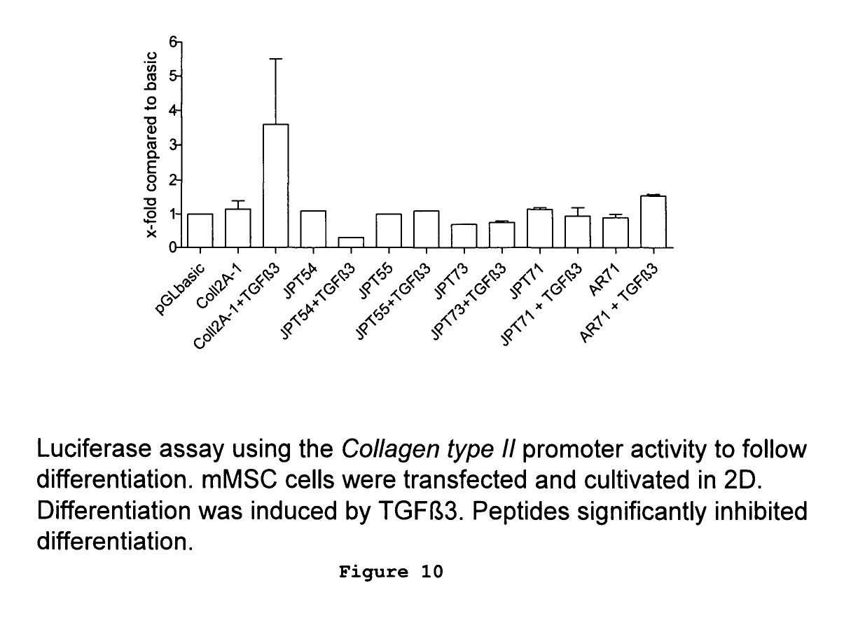

FIG. 10 shows the results of a luciferase assay, measuring the collagen type II promoter activity (=Coll2A-1 luciferase reporter) to follow chondrogenic differentiation. mMSC (murine mesenchymal stem cells) were transfected and cultivated in wells of a 6-well plate. Differentiation was induced by TGFβ3. As can be seen, the peptides in accordance with the present invention significantly inhibited the differentiation.

As MIA is known to be important in chondrogenic differentiation (exemplified in FIG. 9 using siRNA against MIA) inhibition of MIA using the newly defined MIA inhibitory peptides results in inhibition of chondrogenic differentiation. These assays clearly show the strong effect of the inhibitory peptides in 3 independent model systems.

Moreover, reference is made to the examples which are given to illustrate, not to limit the present invention.

### EXAMPLES

### Example 1

Cell Lines and Cell Culture Conditions

The melanoma cell line Mel Im, established from a human metastatic bioptic sample (generous gift from Dr. Johnson, University of Munich, Germany) was used in all experiments. Additionally, main experiments were also conducted using the human cell line Mel Ju and the murine cell line B16, which were derived from metastases of malignant melanoma. All cells were maintained in DMEM (PAA Laboratories GmbH, Cölbe, Germany) supplemented with penicillin (400 U/mL), streptomycin (50 μg/mL), 1-glutamine (300 μg/mL) and 10% fetal calf serum (Pan Biotech GmbH, Aidenbach, Germany) and split in 1:6 ratio every 3 days.

### Protein Analysis In Vitro (Western Blotting)

Protein samples were denaturated at 70° C. for 10 min after addition of reducing and denaturing Roti-Load buffer (Roth, Karlsruhe, Germany) and subsequently separated on sodium dodecyl sulfate 12.75% polyacrylamid gels (SDS-PAGE) (Invitrogen, Groningen, The Netherlands). In the multimerization studies, MIA protein (1 μg) was incubated with AR71 (2.5 μg) overnight at RT before being treated as described above. After transferring the proteins onto a polyvinylidene fluoride (PVDF) membrane (BioRad, Richmond, Va., USA), the membrane was blocked using 3% BSA/PBS for 1 h at RT and incubated with a 1:150 dilution of primary polyclonal rabbit anti MIA antibody (Biogenes, Berlin, Germany) in 3% BSA/PBS overnight at 4° C. After washing in PBS the membrane was incubated with a 1:2000 dilution of an alkaline-phosphate coupled secondary antibody (Chemikon, Hofheim, Germany) for 2 h at RT. Finally, after washing steps, immunoreactions were visualized by nitro blue tetrazolium/5-bromo-4-chloro-3-indolyl phosphate (NBT/BCIP) (Invitrogen, Karlsruhe, Germany) staining.

### Boyden Chamber Invasion Assay

Invasion assays were performed in Boyden Chambers containing polycarbonate filters with 8-μm pore size (Neuro Probe, Gaithersburg, Md., USA) essentially as described previously.¹³ Filters were coated with matrigel, a commercially available reconstituted basement membrane (diluted 1:3 in H₂O; BD Bioscience, Bradford, Mass., USA). The lower compartment was filled with fibroblast-conditioned medium used as a chemo attractant. Mel Im melanoma cells were harvested by trypsinization for 2 min at RT, resuspended in DMEM without FCS at a density 2.5×10⁴ cells/mL, and placed in the upper compartment of the chamber. Except for the control experiment with untreated cells and experiments where cells were only treated with the respective peptide, MIA was added to the cell suspension at a final concentration of 200 ng/mL. Peptide AR71 (sequence: Ac-FHWRYPLPLPGQ-NH₂) was used at a final concentration of 1 μM. MIA expressing murine B16 melanoma cells stably co-transfected with Sig-AR71-HisTag containing pCMX-PL1 vector²⁷ and an antibiotic resistance comprising plasmid (pCDNA3), and the respective mock control were also investigated for their ability to migrate. Therefore, cells were harvested by trypsinization for 2 min at RT, resuspended in DMEM without FCS at a density 2.5×10⁴ cells/mL, and placed in the upper compartment of the chamber. After incubation at 37° C. for 4 h filters were removed. Cells adhering to the lower surface of the filter were fixed, stained, and counted. Experiments were carried out in triplicates and repeated at least three times.

Coating of Well Plates with MIA-Biotin

Black, streptavidin coated 96 well plates (from Greiner Bio-one, Frickenhausen, Germany) were coated with MIA-Biotin as described previously.^{7,14} An uncoated control lane was sealed with adhesive film to prevent contamination. The MIA-Biotin coated plate was used for measurements immediately.

### Polarization Assay Setup

All measurements were performed at RT on a Polarstar Optima microplate reader (BMG Labtech, Offenburg, Germany). A 390-10 nm bandpass filter was used for excitation while a 520 nm longpass filter was used for the emission light. Even though the extinction coefficient is higher at longer wavelengths, the inventors chose a shorter excitation wavelength as this led to higher polarization values. MIA-Ru(bpy)₃ was prepared and tested for functional activity as described previously.¹⁴ A MIA-Ru(bpy)₃ concentration of 55 fM was used in all experiments. A solution volume of 250 μL per well was found to give a low standard deviation with high signal intensity. All measurements were performed in DPBS without calcium or magnesium (PAN Biotech GmbH, Aidenbach, Germany). Addition of components to the wells was done in the following order: inhibitory peptide, buffer, MIA-Ru(bpy)₃. Owing to different reaction kinetics, measurements were performed every 5 min over a 30 min period. Polarization values are reported relative (P/P₀) to the value of free MIA-Ru(bpy)₃ in solution in a well not treated with MIA-biotin. All reported values are an average of three independent measurements.

### Cloning Strategy

Signal-AR71-HisTag pCMX-PL1-plasmid construction: The Signal-AR71-HisTag pCMX-PL1 expression plasmid was created by PCR amplification of the human hydrophobic signal-peptide sequence, responsible for transport into the endoplasmic reticulum, from a Signal-MIA containing expression plasmid using the MJ Research PTC-200 Peltier Thermo Cycler (BioRad, Munich, Germany). The HisTag sequence was inserted at the C-terminal end of the AR71 peptide using the primers 5′-GAC GAA TTC ATG GCC CGG TCC CTG GTG-3′ and 5′-GAC AAG CTT TCA GTG ATG GTG ATG GTG ATG CTG GCC GGG CAA GGG CAA GGG GTA TCT CCA GTG GAA CCT GAC ACC AGG TCC GGA GAA-3′. After amplification of the Signal-AR71-HisTag fragment, the PCR product was digested with EcoRI and HindIII (NEB, Frankfurt, Germany) The insert was purified by gel extraction (Qiagen, Hilden, Germany) and cloned into the EcoRI and HindIII sites of the eukaryotic expression vector pCMX-PL1 which was previously purified and prepared for ligation using T4-Ligase (NEB, Frankfurt, Germany).²⁷ After transformation in DH10β cells (NEB, Frankfurt, Germany) according to the manufacturer's instructions, the plasmid was isolated using the MIDI Kit (Qiagen, Hilden, Germany) and quantified by a gene quant II RNA/DNA Calculator (Pharmacia Biotech, Nümbrecht, Germany). The sequence of the PCR-generated clone was confirmed by DNA sequencing.

### Stable Transfection of Murine B16 Melanoma Cells

For transfection, 1.5×10⁵ cells/mL were seeded in 6-well plates (Corning Omnilab, Munich, Germany) and cultured in 2 mL of Dulbecco's modified Eagle's medium (PAA, Cölbe, Germany) with 10% fetal calf serum (Pan, Aidenbach, Germany). Cells were transfected with 0.8 μg of the respective control or His-tagged AR71 containing pCMX-PL1 vector and 0.2 μg pcDNA3 providing geneticin (Invitrogen, Karlsruhe, Germany) resistance using the LipofectaminPlus (Invitrogen, Karlsruhe, Germany) method according to the manufacturer's instructions. After selection of cells comprising antibiotic resistance the inventors confirmed expression and localization of AR71 peptide on mRNA and protein level by PCR and immunofluorescence, respectively.

### Recombinant Expression of MIA and Mutant Forms

In vitro protein expression reactions of recombinant human MIA and its mutants were performed with the Rapid Translation System RTS 500 E. coli HY Disulfite Kit (Roche, Mannheim, Germany) according to the manufacturer's instructions. All reactions were carried out over night at 30° C. or 25° C. with efficient stirring in the RTS 500 instrument. MIA mutants were checked for correct folding and function as previously described.¹³

### NMR Spectroscopy

All spectra were recorded at 300 K and pH 7 on a Bruker DRX600 spectrometer equipped with a pulsed field gradient triple resonance probe. Water suppression in experiments recorded on samples in H₂O was achieved by incorporation of a Watergate sequence into the various pulse sequences.²⁸⁻³⁰ 2D ¹H-¹⁵N HSQC spectra with reduced signal loss due to fast exchanging protons were recorded using procedures described previously.³¹ All spectra were processed with NMRPipe and analyzed with NMRView.³²⁻³³ Data handling was performed with NMRView. Structure visualisation and superimpositions were done with PyMol (Delano, W. L., The PyMol Molecular Graphics System (2002) Delano Scientific, Palo Alto, Calif., USA).

### Dimer Model

The PreBI modelling software (http://pre-s.protein.osaka-u.ac.in/prebi/) was used together with the published X-ray structure of MIA (PDBid: 1I1J) for the prediction of the putative dimer interface. Employing the monomeric NMR structure of MIA (PDBid: 1HJD) together with the interface information obtained in the previous step a three-dimensional model of the dimeric complex was calculated. Computations were performed using the data driven protein-protein docking program HADDOCK (Dominguez, C., Boelens, R., Bonvin, A. M. J. J. HADDOCK: A Protein-Protein Docking Approach Based on Biochemical or Biophysical Information (2003) J. Am. Chem. Soc. 125, 1731-1737).

### Protein Binding Studies

The NMR titration of MIA with AR71 consisted of monitoring changes in chemical shifts and line widths of the backbone amide resonances of uniformly ¹⁵N-enriched MIA samples as a function of ligand concentration.³⁴⁻³⁷

### In Vivo Metastasis Assay

To determine the effect of peptide AR71 on the metastatic potential of murine B16 melanoma cells in vivo, a previously developed mouse metastases model was used.¹⁹ 1×10⁵ cells of the AR71-HisTag expressing B16 cell clone AR71-His K4 as well as the corresponding mock control cells were injected into the spleen of mice (n=8 for mock control cells as well as for AR71-HisTag K4 cells, respectively). After nine days, mice were sacrificed, the livers were resected and the number and size of visible black tumor nodules on the surface of the livers was noticed. Tissues were fixed in formalin and afterwards paraffin embedded sections were hematoxylin and eosin stained for histological analysis.

Additionally, 1×10⁵ wt mouse melanoma B16 cells suspended in a solution containing AR71 (1 mg/mL) and 0.9% NaCl, or NaCl alone for the control mice, respectively, were injected into the spleen of each animal (n=8 for treated mice, as well as for control without AR71). Peptide AR71 was injected i.v. (50 μg every 24 h). After nine days, the mice were sacrificed and the livers were excised. Following formalin fixation, tissues were embedded in paraffin. Afterwards, sections were prepared and stained using hematoxylin and eosin before being subjected to histological analysis.

### Immunofluorescence Assays

5×10⁵ murine B16 melanoma cells were grown in a 4-well chamber slide (Falcon, BD Bioscience, Heidelberg, Germany). After stable transfection with a Sig-AR71-HisTag containing expression plasmid and the respective pCMX-PL1 mock control, cells were incubated for 48 h at 37° C. and 8% CO₂. Afterwards, cells were washed and fixed using 4% paraformaldehyde in 0.1 M phosphate-buffered saline (PBS) for 15 min. After permeabilization of cells, blocking of non-specific binding sites with blocking solution (1% BSA/PBS) for 1 h at 4° C. was performed. Cells were incubated with primary antibodies rabbit anti-MIA (Biogenes, Berlin, Germany) and mouse anti-HisTag (BD Bioscience, Pharmingen, Germany) at a concentration of 1 μg/mL at 4° C. for 2 h. After rinsing with PBS 5 times, cells were first covered with a 1:200 dilution of the secondary antibody TRITC anti-mouse (TRITC-conjugated donkey anti-mouse antibody, Jackson Immuno Research Laboratories, West Grove, Pa., USA) and FITC anti-rabbit (FITC-conjugated polyclonal swine anti rabbit immunoglobulin, DakoCytomation, Glostrup, Denmark) in PBS at 4° C. for 1 h, respectively. Following incubation with primary antibodies, cells were washed with PBS and coverslips were mounted on slides using Hard Set Mounting Medium with DAPI (Vectashield, H-1500, Linearis, Wertheim Germany) and imaged using an Axio Imager Zeiss Z1 fluorescence microscope (Axiovision Rel. 4.6.3) equipped with an Axio Cam MR camera. Images were taken using 63× oil immersion lenses.

### Statistical Analysis

In the bar graphs, results are expressed as mean±S.D. (range) or percent. Comparison between groups was made using the Student's unpaired t-test. A p-value <0.05 was considered as statistically significant (ns: not significant, *: p<0.05, **: p<0.01, ***: p<0.001). All calculations were made using the GraphPad Prism Software (GraphPad Software, Inc., San Diego, USA).

### Micromass Assay

For analysis of chondrogenesis human MSC differentiation was performed in high density culture. For this, 3×10⁵ cells were seeded into each well of a six-well plate. Cells were cultured for the indicated period in induction medium including DMEM (PAA), high glucose (Sigma), 20% fetal calf serum (FCS, PAN Biotech GmbH), MEM Vitamins (Invitrogen), penicillin (100 U/ml), streptomycin (10 μg/ml) (both Sigma), Amphotericin B (2.5 μg/ml) (PAN Biotech GmbH), 0.1 μM dexamethasone, 1 mM sodium pyruvate, 0.17 mM ascorbic acid-2-phosphate, 0.35 mM proline (all Sigma), insulin (5 μg/ml), transferring (5 μg), selenious acid (5 ng) (ITS Premix, Becton Dickinson) and 10 ng/ml human TGF-β3 (R&D Systems). The medium was changed every second day. All groups were done in triplicate. Each experiment was repeated three times. Analysis was performed on RNA level.

### Hanging Drop Assay

To generate spheroids the hMSC were detached from the culture flask by adding 1 ml Trypsin-EDTA (Provitro). After incubation for a 5 minutes 1 ml neutralizing solution (Provitro) and 8 ml HMSC proliferation medium were added. After centrifugation for 4 min at 1200 rpm and resuspension in Incomplete Chondrogenesis Induction Medium (DMEM with glucose (4.5 g/L), penicillin (400 units/ml) streptomycin (50 μg/ml), L-glutamine (300 μg/ml), sodium pyruvate (1 mM), L-ascorbic acid 2-phosphate (0.17 mM), L-proline (0.35 nM), dexamethasone (1 μM) and ITS premix (BD Biosciences, Heidelberg, Germany)), the cells were counted and adjusted to 50,000 cells/ml. 20% methocel (6 g methyl cellulose (Sigma-Aldrich, Munich, Germany), 250 ml basal medium)) was added and 25 μl of the cells suspension were dropped onto the cover of a 9 mm petri dish. For each experimental condition 10 drops were used. The petri dish was filled with PBS and the cover dish was inverted and incubated for 72 h under a humidified atmosphere of 8% CO₂ at 37° C. for 1, 2, 4 and 7 d followed by RNA isolation or alcian blue staining. For treatment, TGF-beta3 (10 ng/ml, Biomol, Hamburg, Germany) was added. At day 4 NA was extracted and analysed. The experiment was repeated 4 times.

### Luciferase Assay

For transient transfections 2×10⁵ mMSC were seeded into each well of a six-well plate and transfected with 0.5 μg plasmid DNA (Col2A1 LUC) using the Lipofectamine plus method (Gibco, Karlsruhe, Germany) according to the manufacturer's instruction. Cells were cultured in induction medium (see above) including 10 ng/ml human TGF-β3 (R&D Systems) to induce chondrogenic differentiation. The cells were lysed 24 h after transfection and the luciferase activity in the lysate was quantified by a luminometric assay (Promega Corp., Madison, USA). Transfection efficiency was normalized according to renilla luciferase activity by cotransfecting 0.1 μg of the plasmid pRL-TK (Promega, Mannheim, Germany). All transfections were repeated at least three times. The COL2A1 LUC reporter construct (Tan et al., 2003) kindly provided by Linda J. Sandell was used. This is a reporter gene construct carrying the Collagen Type II promoter.

Tan L, Peng H, Osaki M, Choy B K, Auron P E, Sandell L J et al., Egr-1 mediates transcriptional repression of COL2A1 promoter activity by interleukin-1beta. J Biol. Chem. 2003; 278: 17688-17700

### Example 2

### MIA Protein is Functionally Active as a Dimer

Although MIA was thought to act as a monomer, recent data suggests that, as detailed below, the active form of the protein consists of a dimer. Using the PreBI modelling software (http://pre-s.protein.osaka-u.ac.ip/prebi/) for the prediction of the putative dimer interface together with the HADDOCK protein-protein docking program (Dominguez, C., Boelens, R., Bonvin, A. M. J. J. HADDOCK: A Protein-Protein Docking Approach Based on Biochemical or Biophysical Information (2003) J. Am. Chem. Soc. 125, 1731-1737), the inventors obtained a model of the MIA dimer comprising a head to tail linkage (FIG. 1A). The dimerization interfaces are located around cysteine 17, serine 18, tyrosine 47, glycine 61, glycine 66, aspartate 67, leucine 76, tryptophan 102, aspartate 103, cysteine 106, valine 64, tyrosine 69, aspartate 87, and lysine 91 in the first monomer participating in dimerization, wherein the following residues appear to be the most prominent candidates for interface formation: cysteine 17, serine 18, tyrosine 47, glycine 61, glycine 66, aspartate 67, leucine 76, tryptophan 102, aspartate 103 and cysteine 106. In the second monomer, which participates in the dimer formation, different residues are involved, due to the afore-mentioned head-to-tail-linkage. Accordingly, in the second monomer, the interface to the other (first) monomer is formed by residues of the second monomer selected from glycine 54, leucine 58, phenylalanine 59, alanine 7, lysine 53, arginine 55, arginine 57, arginine 85 and lysine 94. In this second monomer, the most prominent residues involved in the formation of the interface are alanine 7, lysine 53, arginine 55, arginine 57, arginine 85 and lysine 94.

In addition, Western blot analysis of MIA also demonstrates that apart from the monomeric species dimers exist.¹⁴ The inventors, therefore, aimed to investigate the physiological relevance of MIA dimers and the possible correlation between dimerization and functional activity. Having identified the most likely positions of the dimerization interfaces, mutants of MIA were tested for their capability to form dimers by Western blot analysis (FIG. 1B). MIA mutants were expressed in an in vitro transcription/translation system. All mutants showed correct folding as evidenced by a MIA-ELISA and were selected as not carrying a mutation in the dimerization regions, apart from G61R.¹³ Recombinant wt MIA and all mutants clearly show a dimer band except for G61R. Interestingly, all mutants but G61R are functionally active in Boyden chamber invasion assays, as presented in FIG. 1C. MIA wt (RTS) and mutants D29G/Y69H, V46F/S81P, T89P and K91N can exhibit this effect to the same extent while MIA mutant G61R completely loses activity. The sites of mutations not affecting functional activity (FIG. 1D, depicted by amino acid labels D29, S81 etc.) are located outside the dimerization regions, whereas G61R (FIG. 1D, depicted by G61) is buried in the dimerization cleft (depicted by the darker grey are in the left panel) in close proximity to the distal loop.

### Peptide AR71(=SEQ ID NO:47) Prevents MIA Protein Dimerization

The inventors then aimed to identify peptides inhibiting MIA dimerization in a newly developed heterogeneous transition-metal based fluorescence polarization (HTFP) assay.¹⁴ First, MIA-MIA interaction was confirmed using this assay. Here, the inventors immobilized a MIA-biotin conjugate in a streptavidin-coated well plate and added MIA labelled with the luminescent transition-metal complex Ru(bpy)₃. As depicted in FIG. 2A, a significant increase in FP signal in the wells coated with MIA-biotin was observed compared to control wells not functionalized with MIA-biotin. This was attributed to the severely restricted rotational mobility of MIA-Ru(bpy)₃ bound to the immobilized MIA-biotin.

The inventors then screened peptides, previously identified by phage display and known to generally bind to MIA¹¹, for their potential to prevent MIA dimerization and induce dissociation of already existing protein dimers using the HTFP assay. As shown in FIG. 2A, peptide AR71 (sequence: Ac-FHWRYPLPLPGQ-NH₂=amidated SEQ ID NO:47) was found to be particularly potent in dissociating MIA dimers which led to a decrease in FP signal due to increased rotational diffusion of the dissociated monomeric MIA-Ru(bpy)₃. This effect of AR71 was confirmed by Western Blot analysis (FIG. 2B). Preincubation of MIA with 1 μM peptide AR71 leads to a strong reduction of the dimer bands compared to the control lane or other MIA-binding peptides used (AR68, AR69).

To prove that AR71 functionally inhibits MIA, Boyden chamber invasion assays were performed (FIG. 2C). In these in vitro experiments, MIA interferes with the attachment of cells to matrigel, as reflected by a decrease in cell invasion. After external treatment with MIA, invasion of Mel Im cells is significantly reduced about 40% to 50% compared to untreated control cells. Pre-incubation of MIA with the inhibitory peptide AR71 results in a complete neutralization of the effect caused by MIA, as reflected in the number of invaded cells. Treatment of cells with peptide AR71 alone does not influence the migratory behaviour of melanoma cells.

MIA Interacts with AR71

After demonstrating the potential of AR71 to inhibit MIA function in in vitro models, the inventors could show by multidimensional NMR spectroscopy that MIA binds to this peptide ligand. In addition, the potential binding site of AR71 was identified using ¹⁵N labeled MIA and unlabeled peptide. By using increasing amounts of AR71 peptide, the induced chemical shift changes of the MIA ¹H^{N} and ¹⁵N^{H} resonances were classified according to the degree of the combined chemical shift perturbations. Further analysis of the solvent accessibility (with a threshold of 20%) and cluster analysis of the residues effected by peptide binding reveals that the binding interface between two monomers comprises residues C17, S18, Y47, G61, G66, D67, L76, W102, D103 and C106 of MIA in one of the monomers (FIG. 3A). It can therefore be assumed that the peptide predominantly binds to the binding site depicted on the left side of FIG. 3A, whereas the opposite side of the molecule most probably does not participate in binding.

After stably transfecting B16 mouse melanoma cells with a secretion-signal containing AR71-HisTag construct (Sig-AR71-HisTag), the inventors first analysed expression and localization of endogenous AR71-HisTag peptide. Co-staining of MIA protein and AR71-HisTag revealed a colocalization in close proximity to the nucleus. Immunofluorescence studies show the localization of MIA (FIG. 3Ba) and AR71-HisTag (for demonstrating colocalization with MIA (see FIG. 3Bb and white arrows in FIG. 3Bc). The excess of MIA not colocalized with AR71 is due to internalization of exogenous MIA protein by the melanoma cells.¹⁵ FIG. 3Bd shows the corresponding mock control.

### Effect of MIA Inhibitory Peptide AR71 on Formation of Metastases In Vivo

MIA expression levels of malignant melanoma cells strictly correlate with a highly invasive phenotype in vitro and in vivo.¹⁶⁻¹⁸ Further, in vivo studies have demonstrated the strong contribution of MIA for melanoma cell invasion and migration.⁴⁻⁵

In order to assess the ability of peptide AR71 to inhibit the formation of metastases by generating inactive MIA monomers in vivo, a previously developed metastasis assay was employed.¹⁹ In this assay, melanoma cells metastasize from the primary tumor in the spleen via the portal vein into the liver. Nine days after injection of the cells into the spleen, the mice were sacrificed, the livers were resected and tissue sections were prepared. Here, the inventors used the stably transfected murine B16 melanoma cells with a Sig-AR71-HisTag containing construct. In vitro analysis by Boyden chamber assay confirmed that migration is drastically reduced in Sig-AR71-HisTag expressing cell clones compared to mock control cells (FIG. 4A). The interference of AR71-HisTag with MIA-MIA interaction was also confirmed in the HTFP assay using wells coated with MIA-biotin (data not shown). Subsequently, a Sig-AR71-HisTag clone as well as a corresponding mock control was injected into the spleen of C57B16 mice, respectively. Histological analysis of haematoxylin and eosin stained liver sections revealed that mice being injected with Sig-AR71-HisTag clones comprised significantly fewer metastases than the mock control (FIG. 4B). Four representative histological liver sections (hematoxylin and eosin stained) of mice injected with the B16 mock control or mice injected with the Sig-AR71-HisTag expressing cell clone, respectively, are shown in FIG. 4C. Black arrows indicate the small metastases in the mock control which are exceedingly reduced in the liver of mice injected with the Sig-AR71-HisTag expressing cell clone. No adverse effects of AR71 on other organs and tissues were observed.

These results prompted the inventors to investigate whether AR71 peptide could also reduce the formation of metastases when given as an i.v. administration treatment. Therefore, wild type murine B16 melanoma cells were injected into the spleen of C57B16 mice with the mice being subsequently treated with i.v. injections of AR71 (50 μg every 24 h). After nine days, the mice were sacrificed, the livers were resected and again tissue sections were prepared. Histological analyses revealed a significant reduction of the average number of metastases in the liver of mice treated with AR71 compared to the liver of untreated control mice, as shown in figure FIG. 4D. Four representative histological liver sections (hematoxylin and eosin stained) of untreated and treated mice, respectively, are shown in FIG. 4E. Again no adverse effects on other organs and tissues were observed.

### Example 3

### Peptides SEQ ID NO:1-45 are Potent Inhibitors of Dimer Formation of MIA

Peptides SEQ ID NO:1-9 were subjected to an HTFP assay as described above, together with peptide AR71, and from FIG. 5 it can be seen that these peptides according to the present invention show a stronger interference with MIA-interaction than SEQ ID NO:46 and 47 and therefore prevent MIA-dimer formation in a stronger fashion. From a mere comparison of these sequences with SEQ ID NO:46 and 47, this was not to be expected and therefore is a surprising finding. The peptides in accordance with the present invention exhibit a significant MIA inhibitory effect in the HTFP assay. As reflected by the HTFP assay, the inhibitory peptides according to the present invention promote the dissociation of MIA protein aggregates or protein dimers. Based on the results achieved with SEQ ID NO:47 (=AR71), it is therefore to be expected that the peptides in accordance with the present invention also prevent the metastasis of malignant melanoma by inhibiting MIA protein dimerization/aggregation.

### Example 4

Peptides were analysed in three independent assays to define their effect on melanoma cell invasion as well as chondrogenic differentiation. FIG. 7 demonstrates that interference of MIA with cell attachment to matrigel results in a decrease in cell invasion; after external treatment with MIA invasion of Mel Im cells is significantly reduced about 40% to 50% compared to untreated control cells. Pre-incubation of MIA with the respective inhibitory peptide results in a neutralization of the MIA effect. FIG. 6 demonstrates the mechanistic mode of action of inhibition of MIA by preventing dimerization.

Use of the peptides in accordance with the present invention resulted in efficient inhibition of chondrogenic differentiation, a process known to be strongly depending on MIA activity. FIG. 8 shows in a micromass assay that Sox9 expression as marker for chondrocytic differentiation is induced after treatment of the cells with TGFβ3. MIA is an important regulator of chondrogenic differentiation after induction by TGFβ3. Using the inhibitory peptides, chondrocytic differentiation by TGFβ3 was strongly inhibited confirming the strong effect of the peptides on MIA activity. Similarly, FIG. 9 shows the results of a hanging drop assay. Accordingly, the peptides in accordance with the present invention, inhibit the induction of Aggrecan, collagen type II and Sox9 during differentiation after day 4. The effects observed for the peptides in accordance with the present invention, in this example JPT55 (SEQ ID NO:9) and JPT73 (SEQ ID NO:5), are similar to those results obtained when using siRNA to inhibit MIA expression. Using a further model system, FIG. 10 shows the results of a luciferase assay, measuring the collagen type II promoter activity (=Coll2A-1 luciferase reporter) to follow chondrogenic differentiation. mMSC (murine mesenchymal stem cells) were transfected and cultivated in wells of a 6-well plate. Differentiation was induced by TGFβ3. As can be seen, the peptides in accordance with the present invention significantly inhibited the differentiation. To conclude, the peptides claimed show a strong inhibition of MIA-mediated chondrogenic differentiation. As MIA is known to be important in chondrogenic differentiation (exemplified in FIG. 9 using siRNA against MIA) inhibition of MIA using the newly defined MIA inhibitory peptides results in inhibition of chondrogenic differentiation. These assays clearly show the strong effect of the inhibitory peptides in 3 independent model systems.

### REFERENCES

1 Bosserhoff, A. K. et al. Melanoma-inhibiting activity, a novel serum marker for progression of malignant melanoma. Cancer Res 57, 3149-3153 (1997).
2 Blesch, A. et al. Cloning of a novel malignant melanoma-derived growth-regulatory protein, MIA. Cancer Res 54, 5695-5701 (1994).
3 Bosserhoff, A. K. et al. Active detachment involves inhibition of cell-matrix contacts of malignant melanoma cells by secretion of melanoma inhibitory activity. Lab Invest 83, 1583-1594 (2003).
4 Bosserhoff, A. K., Echtenacher, B., Hein, R. & Buettner, R. Functional role of melanoma inhibitory activity in regulating invasion and metastasis of malignant melanoma cells in vivo. Melanoma Res 11, 417-421 (2001).
5 Guba, M. et al. Overexpression of melanoma inhibitory activity (MIA) enhances extravasation and metastasis of A-mel 3 melanoma cells in vivo. Br J Cancer 83, 1216-1222, doi:10.1054/bjoc.2000.1424
   S0007092000914241 [pii] (2000).
6 Schmidt, J., Friebel, K., Schönherr, R., Coppolino, M. G. & Bosserhoff, A. K. Directed, Migration-associated Secretion of Melanoma Inhibitory Activity (MIA) at the Cell Rear is supported by KCNN4 Potassium Channels. Int. J. Cancer (submitted) (2010).
7 Bauer, R. et al. Regulation of integrin activity by MIA. J Biol Chem 281, 11669-11677, doi:M511367200 [pii]
   10.1074/jbc.M511367200 (2006).
8 Stoll, R. & Bosserhoff, A. Extracellular SH3 domain containing proteins—features of a new protein family. Curr Protein Pept Sci 9, 221-226 (2008).
9 Stoll, R. et al. Sequence-specific 1H, 13C, and 15N assignment of the human melanoma inhibitory activity (MIA) protein. J Biomol NMR 17, 87-88 (2000).
10 Stoll, R. et al. Backbone dynamics of the human MIA protein studied by (15)N NMR relaxation: implications for extended interactions of SH3 domains. Protein Sci 12, 510-519, doi:10.1110/ps. 0222603 (2003).
11 Stoll, R. et al. The extracellular human melanoma inhibitory activity (MIA) protein adopts an SH3 domain-like fold. EMBO J 20, 340-349, doi:10.1093/emboj/20.3.340 (2001).
12 Lougheed, J. C., Holton, J. M., Alber, T., Bazan, J. F. & Handel, T. M. Structure of melanoma inhibitory activity protein, a member of a recently identified family of secreted proteins. Proc Natl Acad Sci USA 98, 5515-5520, doi:10.1073/pnas.091601698
   091601698 [pii] (2001).
13 Stoll, R., Lodermeyer, S. & Bosserhoff, A. K. Detailed analysis of MIA protein by mutagenesis. Biol Chem 387, 1601-1606, doi:10.1515/BC.2006.199 (2006).
14 Riechers, A., Schmidt, J., Konig, B. & Bosserhoff, A. K. Heterogeneous transition metal-based fluorescence polarization (HTFP) assay for probing protein interactions. Biotechniques 47, 837-844, doi:000113223 [pii]
   10.2144/000113223 (2009).
15 Schmidt, J. & Bosserhoff, A. K. Processing of MIA protein during melanoma cell migration. Int J Cancer 125, 1587-1594, doi:10.1002/ijc.24508 (2009).
16 Bosserhoff, A. K., Hein, R., Bogdahn, U. & Buettner, R. Structure and promoter analysis of the gene encoding the human melanoma-inhibiting protein MIA. J Biol Chem 271, 490-495 (1996).
17 Bosserhoff, A. K. et al. MIA, a novel serum marker for progression of malignant melanoma. Anticancer Res 19, 2691-2693 (1999).
18 Stahlecker, J. et al. MIA as a reliable tumor marker in the serum of patients with malignant melanoma. Anticancer Res 20, 5041-5044 (2000).
19 Carrascal, M. T. et al. Interleukin-18 binding protein reduces b16 melanoma hepatic metastasis by neutralizing adhesiveness and growth factors of sinusoidal endothelium. Cancer Res 63, 491-497 (2003).
20 Lougheed, J. C., Domaille, P. J. & Handel, T. M. Solution structure and dynamics of melanoma inhibitory activity protein. J Biomol NMR 22, 211-223 (2002).
21 Osborne, J. C., Jr., Bengtsson-Olivecrona, G., Lee, N. S. & Olivecrona, T. Studies on inactivation of lipoprotein lipase: role of the dimer to monomer dissociation. Biochemistry 24, 5606-5611 (1985).
22 Nomura, A. M., Marnett, A. B., Shimba, N., Dotsch, V. & Craik, C. S. One functional switch mediates reversible and irreversible inactivation of a herpesvirus protease. Biochemistry 45, 3572-3579, doi:10.1021/bi0523658 (2006).
23 Pray, T. R., Nomura, A. M., Pennington, M. W. & Craik, C. S. Auto-inactivation by cleavage within the dimer interface of Kaposi's sarcoma-associated herpesvirus protease. J Mol Biol 289, 197-203, doi:10.1006/jmbi.1999.2791
   S0022-2836(99)92791-2 [pii] (1999).
24 Wlodawer, A. et al. Conserved folding in retroviral proteases: crystal structure of a synthetic HIV-1 protease. Science 245, 616-621 (1989).
25 Boggetto, N. & Reboud-Ravaux, M. Dimerization inhibitors of HIV-1 protease. Biol Chem 383, 1321-1324, doi:10.1515/BC.2002.150 (2002).
26 Moser, M. et al. Ultrastructural cartilage abnormalities in MIA/CD-RAP-deficient mice. Mol Cell Biol 22, 1438-1445 (2002).
27 Tatzel, J., Poser, I., Schroeder, J. & Bosserhoff, A. K. Inhibition of melanoma inhibitory activity (MIA) expression in melanoma cells leads to molecular and phenotypic changes. Pigment Cell Res 18, 92-101, doi:PCR212 [pii] 10.1111/j.1600-0749.2005.00212.x (2005).
28 Braunschweiler, L. & Ernst, R. R. Coherence Transfer by Isotropic Mixing—Application to Proton Correlation Spectroscopy. Journal of Magnetic Resonance 53, 521-528 (1983).
29 Davis, D. G. & Bax, A. Simplification of H-1-Nmr Spectra by Selective Excitation of Experimental Subspectra. J. Am. Chem. Soc. 107, 7197-7198 (1985).
30 Shaka, A. J., Lee, C. J. & Pines, A. Iterative Schemes for Bilinear Operators—Application to Spin Decoupling. Journal of Magnetic Resonance 77, 274-293 (1988).
31 Mori, S., Abeygunawardana, C., Johnson, M. O. & van Zijl, P. C. Improved sensitivity of HSQC spectra of exchanging protons at short interscan delays using a new fast HSQC (FHSQC) detection scheme that avoids water saturation. J Magn Reson B 108, 94-98 (1995).
32 Johnson, R. D., Bluemler, P., Rafey, R. & Brodbeck, D. Visualization of Multidimensional NMR Data. Abstr Pap Am Chem S 207 (1994).
33 Delaglio, F. et al. NMRPipe: a multidimensional spectral processing system based on UNIX pipes. J Biomol NMR 6, 277-293 (1995).
34 Berghaus, C., Schwarten, M., Heumann, R. & Stoll, R. Sequence-specific 1H, 13C, and 15N backbone assignment of the GTPase rRheb in its GDP-bound form. Biomol NMR Assign 1, 45-47, doi:10.1007/s12104-007-9013-4 (2007).
35 Schwarten, M., Berghaus, C., Heumann, R. & Stoll, R. Sequence-specific 1H, 13C, and 15N backbone assignment of the activated 21 kDa GTPase rRheb. Biomol NMR Assign 1, 105-108, doi:10.1007/s12104-007-9030-3 (2007).
36 Song, J. & Markley, J. L. NMR chemical shift mapping of the binding site of a protein proteinase inhibitor: changes in the (1)H, (13)C and (15)N NMR chemical shifts of turkey ovomucoid third domain upon binding to bovine chymotrypsin A(alpha). J Mol Recognit 14, 166-171, doi:10.1002/jmr.530 [pii]
   10.1002/jmr.530 (2001).
37 Stoll, R. et al. Chalcone derivatives antagonize interactions between the human oncoprotein MDM2 and p53. Biochemistry 40, 336-344, doi:bi000930v [pii] (2001).

The features of the present invention disclosed in the specification, the claims and/or in the accompanying drawings, may, both separately, and in any combination thereof, be material for realizing the invention in various forms thereof.

1-22. (canceled)

23. A peptide or antibody that binds to melanoma inhibitory activity (MIA) protein and prevents dimerization and/or aggregation thereof, which peptide is not SEQ ID NO:46 or 47, wherein binding of said peptide to MIA protein occurs at a surface of said MIA protein formed by at least three amino acid residues of said MIA protein selected from cysteine 17, serine 18, tyrosine 47, glycine 61, glycine 66, aspartate 67, leucine 76, tryptophan 102, aspartate 103, cysteine 106, valine 64, tyrosine 69, aspartate 87, lysine 91, glycine 54, leucine 58, phenylalanine 59, alanine 7, lysine 53, arginine 55, arginine 57, arginine 85, and lysine 94, preferably cysteine 17, serine 18, tyrosine 47, glycine 61, glycine 66, aspartate 67, leucine 76, tryptophan 102, aspartate 103, cysteine 106, alanine 7, lysine 53, arginine 55, arginine 57, arginine 85 and lysine 94, more preferably cysteine 17, serine 18, tyrosine 47, glycine 61, glycine 66, aspartate 67, leucine 76, tryptophan 102, aspartate 103 and cysteine 106.

24. The peptide or antibody according to claim 23, wherein binding thereof to MIA protein is measured by a heterogeneous transition metal-based fluorescence polarization (HTFP) assay, wherein binding of said peptide or antibody to MIA protein is indicated by a ratio P/P₀, wherein P is the fluorescence polarization signal of an MIA protein labeled with a luminescent transition metal complex in the presence of a substrate-bound MIA protein and in the presence of said peptide or antibody, and P₀ is the fluorescence polarization signal of free MIA protein labeled with said luminescent transition metal complex in the absence of a substrate bound MIA protein and in the absence of said peptide or antibody, wherein the ratio P/P₀ of said peptide or antibody, when determined in a heterogeneous transition metal-based fluorescence polarization (HTFP) assay at a defined concentration of said peptide or antibody, is smaller than P/P₀ of the peptide having the amino acid sequence of SEQ ID NO:47, said P/P₀ of said SEQ ID NO:47 peptide having been determined in a HTFP assay at the same defined peptide concentration, or wherein binding thereof to MIA protein is determined by NMR, preferably heteronuclear NMR.

25. The peptide, according to claim 23, having an amino acid sequence selected from SEQ ID NOs:1-45.

26. The peptide or antibody according to claim 23, that is amidated at its C-terminus or is pegylated.

27. A method of treatment of a cancer, said method comprising administration of the peptide or antibody according to claim 23 to a patient having a cancer.

28. The method according to claim 27, wherein said method of treatment prevents metastasis of said cancer.

29. A method of treatment of a degenerative disorder of cartilage, said method comprising administration of the peptide or antibody according to claim 23 to a patient having a degenerative disorder of cartilage.

30. A nucleic acid encoding the peptide or antibody according to claim 23.

31. A vector or construct comprising the nucleic acid according to claim 30.

32. A cell or tissue comprising the nucleic acid according to claim 30.

33. A pharmaceutical composition comprising the peptide or antibody according to claim 23 or a nucleic acid encoding the peptide or antibody according to claim 23, and a suitable pharmaceutically acceptable carrier.

34. A method of preventing dimerization and/or aggregation of melanoma inhibitory activity (MIA) protein, said method comprising:
exposing a MIA protein to a compound which selectively interacts with and/or binds to a surface of said MIA protein formed by at least three amino acid residues of said MIA protein, said at least three amino acid residues being selected from cysteine 17, serine 18, tyrosine 47,

[symbol]

44. The antibody, according to claim 43, having an amino acid sequence selected from SEQ ID NOs:1-9.

45. The method, according to claim 27, wherein the cancer is selected from melanoma, chondrosarcoma, mamma carcinoma and colon carcinoma.

46. The method, according to claim 29, wherein the degenerative disorder of cartilage is selected from rheumatoid arthritis, degeneration of cartilage in a joint, degenerative disc disease, meniscus tears, anterior crucial ligament (ACL) injury, arthritis, osteoarthritis, psoriatic arthritis, juvenile chronic arthritis, rhizomelic arthritis, rheumatoid poly-arthritis, synovitis and villonodular synovitis.

47. The method, according to claim 37, wherein said peptide has an amino acid sequence selected from SEQ ID NOs:1-9.


SEQUENCE LISTING (48 sequences)

SEQ ID NO: 1 - protein, 3 aa, Artificial Sequence
  Peptide Sequence identified from HTFP assay screening
WHF

SEQ ID NO: 2 - protein, 18 aa, Artificial Sequence
  Peptide Sequence identified from HTFP assay screening
FHWRYPLPLPGQHHHHHH

SEQ ID NO: 3 - protein, 6 aa, Artificial Sequence
  Peptide Sequence identified from HTFP assay screening
WHWRYP

SEQ ID NO: 4 - protein, 12 aa, Artificial Sequence
  Peptide Sequence identified from HTFP assay screening
FHWRYPDPDPGQ

SEQ ID NO: 5 - protein, 3 aa, Artificial Sequence
  Peptide Sequence identified from HTFP assay screening
WWW

SEQ ID NO: 6 - protein, 18 aa, Artificial Sequence
  Peptide Sequence identified from HTFP assay screening
HHHHHHFHWRYPLPLPGQ

SEQ ID NO: 7 - protein, 3 aa, Artificial Sequence
  Peptide Sequence identified from HTFP assay screening
WHW

SEQ ID NO: 8 - protein, 12 aa, Artificial Sequence
  Peptide Sequence identified from HTFP assay screening
FHWRYPGPGPGQ

SEQ ID NO: 9 - protein, 4 aa, Artificial Sequence
  Peptide Sequence identified from HTFP assay screening
FHWH

SEQ ID NO: 10 - protein, 8 aa, Artificial Sequence
  Peptide Sequence identified from HTFP assay screening
KGRGRLFW

SEQ ID NO: 11 - protein, 3 aa, Artificial Sequence
  Peptide Sequence identified from HTFP assay screening
FHW

SEQ ID NO: 12 - protein, 12 aa, Artificial Sequence
  Peptide Sequence identified from HTFP assay screening
FHRRYPDPDPGQ

SEQ ID NO: 13 - protein, 6 aa, Artificial Sequence
  Peptide Sequence identified from HTFP assay screening
FHWWYP

SEQ ID NO: 14 - protein, 12 aa, Artificial Sequence
  Peptide Sequence identified from HTFP assay screening
FHWRYPGPLPGQ

SEQ ID NO: 15 - protein, 12 aa, Artificial Sequence
  Peptide Sequence identified from HTFP assay screening
FHWRYPLPGPGQ

SEQ ID NO: 16 - protein, 12 aa, Artificial Sequence
  Peptide Sequence identified from HTFP assay screening
FHWRYPDPLPGQ

SEQ ID NO: 17 - protein, 12 aa, Artificial Sequence
  Peptide Sequence identified from HTFP assay screening
FHWRYPLPDPGQ

SEQ ID NO: 18 - protein, 12 aa, Artificial Sequence
  Peptide Sequence identified from HTFP assay screening
AHWRYPLPLPGQ

SEQ ID NO: 19 - protein, 12 aa, Artificial Sequence
  Peptide Sequence identified from HTFP assay screening
FAWRYPLPLPGQ

SEQ ID NO: 20 - protein, 12 aa, Artificial Sequence
  Peptide Sequence identified from HTFP assay screening
FWWRYPLPLPGQ

SEQ ID NO: 21 - protein, 12 aa, Artificial Sequence
  Peptide Sequence identified from HTFP assay screening
FHWGYPLPLPGQ

SEQ ID NO: 22 - protein, 12 aa, Artificial Sequence
  Peptide Sequence identified from HTFP assay screening
FHWEYPLPLPGQ

SEQ ID NO: 23 - protein, 12 aa, Artificial Sequence
  Peptide Sequence identified from HTFP assay screening
FHWRYALPLPGQ

SEQ ID NO: 24 - protein, 12 aa, Artificial Sequence
  Peptide Sequence identified from HTFP assay screening
FHWRYPLALPGQ

SEQ ID NO: 25 - protein, 12 aa, Artificial Sequence
  Peptide Sequence identified from HTFP assay screening
FHWRYPLPLAGQ

SEQ ID NO: 26 - protein, 6 aa, Artificial Sequence
  Peptide Sequence identified from HTFP assay screening
FHWRYP

SEQ ID NO: 27 - protein, 12 aa, Artificial Sequence
  Peptide Sequence identified from HTFP assay screening
FHWRAPLPLPGQ

SEQ ID NO: 28 - protein, 12 aa, Artificial Sequence
  Peptide Sequence identified from HTFP assay screening
FHWRYPLPLPGR

SEQ ID NO: 29 - protein, 12 aa, Artificial Sequence
  Peptide Sequence identified from HTFP assay screening
FRWRYPLPLPGQ

SEQ ID NO: 30 - protein, 7 aa, Artificial Sequence
  Peptide Sequence identified from HTFP assay screening
RFHWRYP

SEQ ID NO: 31 - protein, 11 aa, Artificial Sequence
  Peptide Sequence identified from HTFP assay screening
FHWRYPLPLPG

SEQ ID NO: 32 - protein, 8 aa, Artificial Sequence
  Peptide Sequence identified from HTFP assay screening
FHWRYPLP

SEQ ID NO: 33 - protein, 7 aa, Artificial Sequence
  Peptide Sequence identified from HTFP assay screening
FHPRYPL

SEQ ID NO: 34 - protein, 3 aa, Artificial Sequence
  Peptide Sequence identified from HTFP assay screening
FFW

SEQ ID NO: 35 - protein, 3 aa, Artificial Sequence
  Peptide Sequence identified from HTFP assay screening
WFF

SEQ ID NO: 36 - protein, 3 aa, Artificial Sequence
  Peptide Sequence identified from HTFP assay screening
WFW

SEQ ID NO: 37 - protein, 3 aa, Artificial Sequence
  Peptide Sequence identified from HTFP assay screening
WFH

SEQ ID NO: 38 - protein, 3 aa, Artificial Sequence
  Peptide Sequence identified from HTFP assay screening
FHF

SEQ ID NO: 39 - protein, 3 aa, Artificial Sequence
  Peptide Sequence identified from HTFP assay screening
HHF

SEQ ID NO: 40 - protein, 3 aa, Artificial Sequence
  Peptide Sequence identified from HTFP assay screening
FHH

SEQ ID NO: 41 - protein, 3 aa, Artificial Sequence
  Peptide Sequence identified from HTFP assay screening
HHH

SEQ ID NO: 42 - protein, 3 aa, Artificial Sequence
  Peptide Sequence identified from HTFP assay screening
WWF

SEQ ID NO: 43 - protein, 3 aa, Artificial Sequence
  Peptide Sequence identified from HTFP assay screening
WWH

SEQ ID NO: 44 - protein, 3 aa, Artificial Sequence
  Peptide Sequence identified from HTFP assay screening
HWW

SEQ ID NO: 45 - protein, 3 aa, Artificial Sequence
  Peptide Sequence identified from HTFP assay screening
FWW

SEQ ID NO: 46 - protein, 12 aa, Artificial Sequence
  Peptide Sequence identified from HTFP assay screening
FHWHPRLWPLPS

SEQ ID NO: 47 - protein, 12 aa, Artificial Sequence
  Peptide Sequence identified from HTFP assay screening
FHWRYPLPLPGQ

SEQ ID NO: 48 - protein, 107 aa, Homo sapiens
GPMPKLADRKLCADQECSHPISMAVALQDYMAPDCRFLTIHRGQVVYVFSKLKGRGRLFWGGSVQGDYYGDLAARLGYFPSSIVREDQTLKPGKVDVKTDKWDFYCQ